(19) United States
(12) Patent Application Publication
(10) Pub. No.: US 2018/0113110 A1
(43) Pub. Date: Apr. 26, 2018

(54) SAMPLE COLLECTION, MANAGEMENT, AND EVALUATION

(71) Applicant: Connect TechNova Inc, San Diego, CA (US)

(72) Inventors: Kenny Fok, San Diego, CA (US); Niral Bhalodia, San Diego, CA (US); David Diplock, Alpine, CA (US); Eric Sterrett, Poway, CA (US)

(21) Appl. No.: 15/790,274

(22) Filed: Oct. 23, 2017

Related U.S. Application Data
(60) Provisional application No. 62/411,521, filed on Oct. 21, 2016.

(51) Int. Cl. G01N 33/487; G01N 33/483; G01N 33/52; G01N 33/94; B01L 3/00; G01N 35/00

(52) U.S. Cl. CPC G01N 33/4875; G01N 33/483; G01N 33/523; G01N 33/94; B01L 3/502715; G01N 35/00009; G01N 2035/00099; G01N 2035/00108; G01N 2035/00019; B01L 2300/0816; G01N 2035/00227; G01N 2035/00326; G01N 35/00871

(57) ABSTRACT

A medical test system includes: a test cartridge having a sample collection portion, a test substrate, a result indicator, and a reservoir that stores a test solution; and a measurement station including: a reservoir lock that couples to the reservoir; a tester manipulator that couples to the sample collection portion and is able to insert the test substrate into the reservoir portion in order to apply the test solution to the test substrate; and a sensor able to identify a result. A medical test cartridge includes: a tester portion including: a collection element; substrate; and result indicator; and a base portion including: a reservoir housing a test solution. An automated method of evaluating a medical sample includes: receiving a base portion of a test cartridge; securing the base portion using a retention element; receiving a tester portion of the test cartridge; and inserting the tester portion into the base portion.

### CROSS-REFERENCE TO RELATED APPLICATIONS

This application claims priority to U.S. Provisional Patent Application Ser. No. 62/411,521, filed on Oct. 21, 2016.

### BACKGROUND

Many medical professionals utilize and develop rapid accurate test kits for detecting and/or diagnosing the presence of drugs or other contraband, disease or other condition, food safety, environmental pollution, etc.

Such test kits typically utilize test paper to collect a sample and a test solution that is used to evaluate the collected sample. A user may submerge the collected sample into the solution and wait for a result indication (e.g., a color indicator on a portion of the test paper).

Many of the test and evaluation operations may require user intervention, such as collection of the sample, opening a seal on a solution container, submerging the test paper into the opened solution, waiting for a specified elapsed time, comparing the result to a color chart (or other result list) to identify a result, and recording the test result.

Therefore there exists a need for a test kit that automates test collection, evaluation, and recordation of results.

### SUMMARY

Some embodiments may provide a test cartridge. The test cartridge may include a “tester” or “collector” portion and a “base” or “reservoir” portion.

The tester portion may include a sample collection feature. The sample collection feature may be able to collect a sample by swiping the feature across a test subject. The tester portion may further include a result window where a result indicator may be displayed or otherwise provided. The sample collection feature and the result window may be included in a single housing. The housing may hold various test papers and/or other test substrates.

The base portion may include a sealed reservoir that houses a test solution for use in evaluating the collected sample. The base portion may be included in a separate housing than the tester portion. Different embodiments may include different solutions (and/or modify other attributes such as amount used, concentration, etc.).

The tester and base may be able to be coupled together in a first configuration such that the sample collection feature is not exposed (i.e., the sample collection feature may be covered by the base housing). Such a configuration may allow the tester to be removed from the base in order to collect a sample. After a sample is collected, the tester and base may be recoupled in the first configuration.

The tester and base may further be able to be coupled together in a second configuration such that the collected sample (and test substrate) are exposed to the test solution in the reservoir.

The tester and base may include various tabs, slots, receptacles, cavities, etc. that may allow the tester and base to be manipulated by automated components. Such manipulation components may be included in a measurement station of some embodiments.

The measurement station may further include various sensors, processing elements, communication features, etc. that may allow the measurement station to interact with the test cartridge in order to process and evaluate a test. In addition, the measurement station may be able to communicate test results to various external devices (e.g., servers, mother devices, user devices, etc.). Alternatively and/or conjunctively, the measurement station may include various user interface (UI) elements that may indicate a result.

In an exemplary usage scenario, a test cartridge may be inserted into the measurement station. The base portion of the cartridge may be secured in place by the measurement station, allowing the tester portion to be removed. A sample may be collected using the tester portion and, after collecting the sample, the tester portion may be inserted into the base portion held in place by the measurement station. The measurement station may move the tester portion into the base portion such that the sample collection element contacts the test solution.

The measurement station may verify that any test criteria are satisfied (e.g., exposure duration) and then may determine a test result. Such a test result may be determined by evaluating some visual element of the test cartridge in some embodiments. For instance, each of a set of colors may be associated with each of a set of possible test outcomes.

The preceding Summary is intended to serve as a brief introduction to various features of some exemplary embodiments. Other embodiments may be implemented in other specific forms without departing from the scope of the disclosure.

### BRIEF DESCRIPTION OF THE SEVERAL VIEWS OF THE DRAWINGS

The exemplary features of the disclosure are set forth in the appended claims. However, for purpose of explanation, several embodiments are illustrated in the following drawings.

FIG. 1 illustrates a front elevation view of a test cartridge according to an exemplary embodiment;

FIG. 2 illustrates a front elevation view of a tester portion included in the test cartridge of FIG. 1;

FIG. 3 illustrates a front sectional view of a base portion of the test cartridge of FIG. 1;

FIG. 4 illustrates a top plan view of a measurement station according to an exemplary embodiment;

FIG. 5 illustrates a front sectional view of the measurement station of FIG. 4 where the test cartridge of FIG. 1 has been inserted into the measurement station;

FIG. 6 illustrates a front sectional view of the measurement station of FIG. 4 where the tester portion of the cartridge of FIG. 1 has been separated from the base portion;

FIG. 7 illustrates a front sectional view of the measurement station of FIG. 4 where the tester portion of the cartridge of FIG. 1 has been fully inserted into the base portion;

FIG. 8 illustrates a side sectional view of the measurement station of FIG. 4 including a first set of exemplary cartridge retention and manipulation elements;

FIG. 9 illustrates a side sectional view of the measurement station of FIG. 4 including the first set of exemplary cartridge retention and manipulation elements after the cartridge of FIG. 1 has been inserted;

FIG. 10 illustrates a side sectional view of the measurement station of FIG. 4 including a second set of exemplary cartridge retention and manipulation elements;

FIG. 11 illustrates a side sectional view of the measurement station of FIG. 4 including the second set of exemplary cartridge retention and manipulation elements after the cartridge of FIG. 1 has been inserted;

FIG. 12 illustrates a schematic block diagram of a system that provides sample management and evaluation according to an exemplary embodiment;

FIG. 13 illustrates a flow chart of an exemplary sample management and evaluation process implemented by the system and devices of FIG. 1-FIG. 12; and

FIG. 14 illustrates a schematic block diagram of an exemplary computer system used to implement some embodiments.

### DETAILED DESCRIPTION

The following detailed description describes currently contemplated modes of carrying out exemplary embodiments. The description is not to be taken in a limiting sense, but is made merely for the purpose of illustrating the general principles of some embodiments, as the scope of the disclosure is best defined by the appended claims.

Various features are described below that can each be used independently of one another or in combination with other features. Broadly, some embodiments generally provide a test kit for sample collection, management, and evaluation.

A first exemplary embodiment provides a medical test system comprising: a test cartridge having a sample collection portion, a test substrate, a result indicator, and a reservoir portion that stores a test solution; and a measurement station comprising: a reservoir lock that couples to the reservoir portion of the test cartridge; a tester manipulator that couples to the sample collection portion of the test cartridge and is able to insert the test substrate into the reservoir portion in order to apply the test solution to the test substrate; and at least one sensor able to identify a result by evaluating the result indicator.

A second exemplary embodiment provides a medical test cartridge comprising: a tester portion including: a test collection element; a test substrate; and a result indicator; and a base portion coupled to the tester portion, the base portion comprising: a reservoir that houses a test solution.

A third exemplary embodiment provides an automated method of evaluating a medical sample, the automated method comprising: receiving a base portion of a test cartridge; securing the base portion using a retention element; receiving a tester portion of the test cartridge; and inserting the tester portion into the base portion.

Several more detailed embodiments are described in the sections below. Section I provides a description of a test cartridge and measurement station provided by some embodiments. Section II then describes a hardware architecture of some embodiments. Next, Section III describes methods of operation used by some embodiments. Lastly, Section IV describes a computer system which implements some of the embodiments.

### I. Test Cartridge and Measurement Station

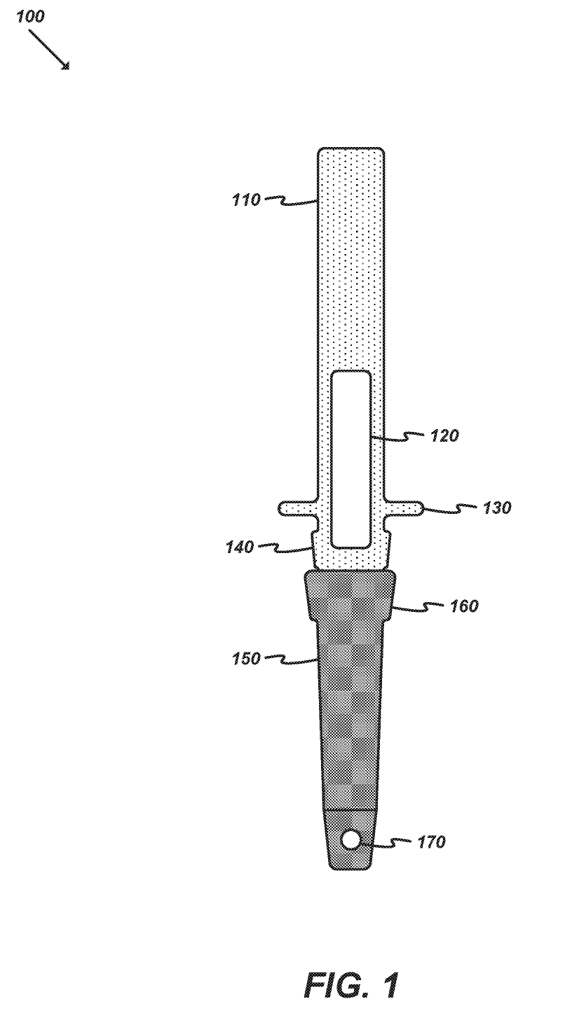

FIG. 1 illustrates a front elevation view of a test cartridge 100 according to an exemplary embodiment. As shown, the cartridge may include a tester portion 110 that may include a result window 120 (or result “indicator”), various tabs, slots, notches, etc. 130, and various coupling elements 140. In addition, the cartridge 100 may include a base portion 150 that includes various cavities, notches, tabs, etc. 160, and a receptacle 170. The cartridge 100 may include a housing made from rigid materials such as plastic, metal, glass, etc.

The result window 120 may simply be an opening in the tester housing 110 or may include various transparent or semi-transparent materials.

The tabs 130 may include protruding elements as shown, and/or various other features that are able to be engaged by a user and/or cartridge manipulation features of some embodiments. Different embodiments may have different numbers, sizes, arrangements, shapes, etc. of tabs than shown.

The coupling elements 140 may allow the tester 110 to be coupled to the base 150. The coupling elements 140 may generally have complementary cavities 160 in the base 150. In addition, various other coupling elements may be included that may engage or partially engage the base cavities 160. For instance, some embodiments may include coupling elements associated with a pre-test configuration as shown in FIG. 1.

The cavities 160 may allow the tester 110 to be inserted into the base 150 until the coupling elements 150 are aligned with the cavities 160.

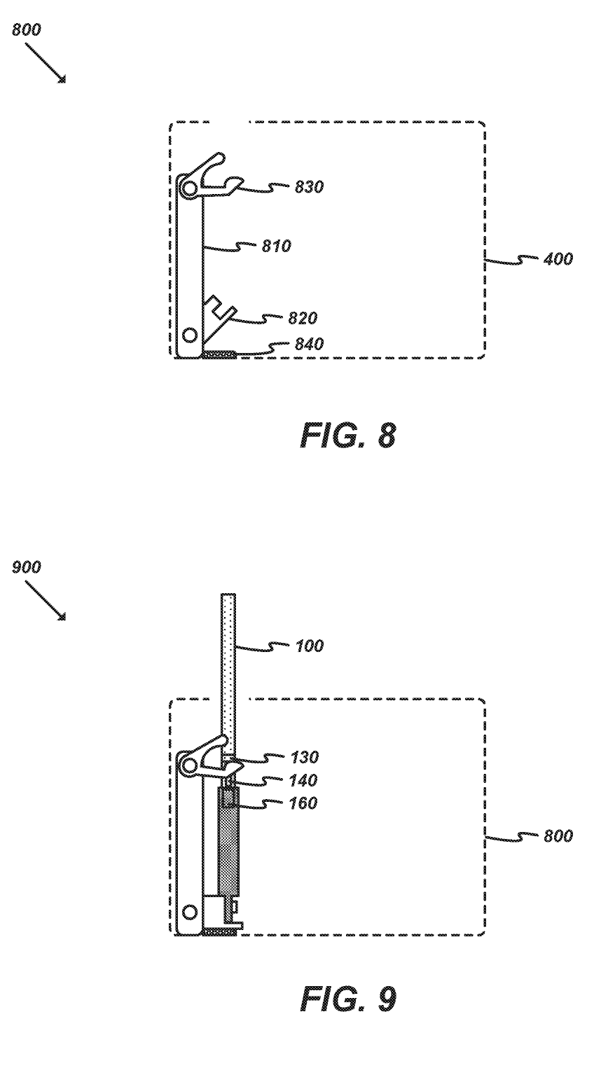

The receptacle 170 may allow the base 150 (and the tester 110 when coupled to the base 150) to be coupled to a measurement station. The receptacle 170 in this example is a through hole, but different embodiments may include different notches, slots, cavities, tabs, etc. that may interface with different types of retention elements utilized by the measurement station. Various example retention elements will be described in more detail in reference to FIG. 8-FIG. 11 below.

In the example of FIG. 1, the tester 110 is partially inserted into the base 150, such that any test solution reservoir remains intact and any test paper is not exposed to the test solution. Cartridges 100 may typically be provided for use in such a state.

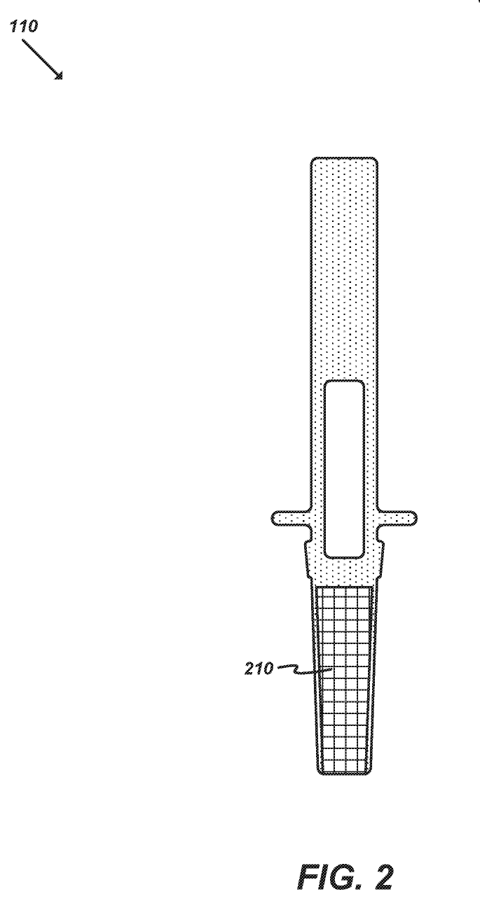

FIG. 2 illustrates a front elevation view of the tester portion 110 included in the test cartridge 100. As shown, the tester portion 110 may include, in addition to the features described above, a collection element 210.

The collection element 210 may include test paper and/or other appropriate collection materials. The collection element may be exposed when the tester portion 110 is removed from the base 150. In this example, the collection element 210 is shown as a portion of the tester 110 end. The collection element 210 may extend to all or a portion of the window 120 such that a test result may be identified.

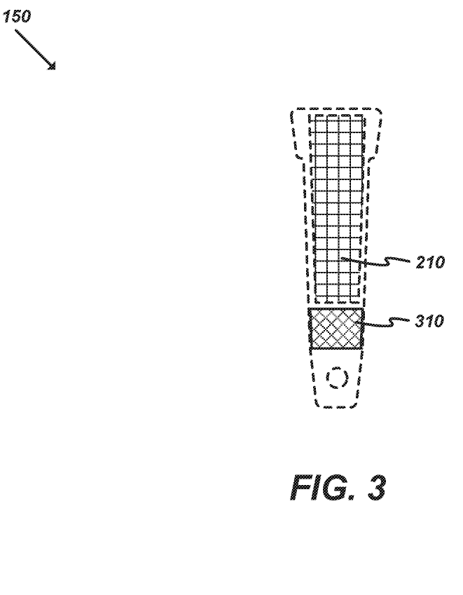

FIG. 3 illustrates a front sectional view of the base portion 150 of the test cartridge 100. As shown, the base 150 may include a reservoir portion 310 that accommodates a test solution included in the cartridge 100.

In this example arrangement, similar to the example of FIG. 1, the collection element 210 is not exposed to the test solution reservoir 310, which remains intact. The collection element 210 is included to show the relationship to the reservoir 310, but the collection element may be included in the tester portion 110 rather than the base 150.

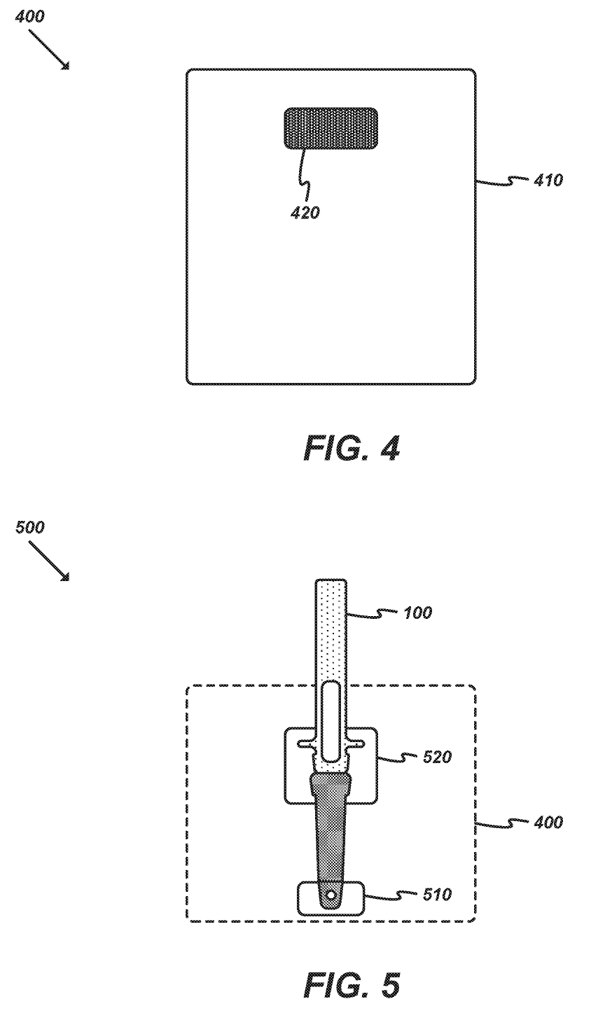

FIG. 4 illustrates a top plan view of a measurement station 400 according to an exemplary embodiment. In this example, the base may include a generally rectangular housing 410, and a test cartridge slot 420. One of ordinary skill in the art will recognize that the base 400 may include various other appropriate elements, such as UI elements (e.g., buttons, LED indicators, touchscreens, keypads, speakers, etc.), mounting elements, etc.

FIG. 5 illustrates a front sectional view of the measurement station 400 where the test cartridge 100 has been inserted into the measurement station 400. As shown, the measurement station may include a first manipulation element 510 (or “retention element”, or “lock”), and a second manipulation element 520. In this example, the first element 510 may serve as a reservoir lock. The first element may also be able to manipulate the reservoir 150 or cartridge 100 when the tester 110 is attached. The second element 520 in this example may serve as a manipulator for the tester 110 (or the cartridge 100 when the base 150 is attached).

Such retention and manipulation elements 510 and 520 may include various tabs, slots, recesses, protrusions, etc. that may be able to interact with features of the cartridge 100. In addition, such features may be associated with various actuators, motors, sensors, etc. that may be able to manipulate and/or otherwise control the manipulation elements 510-520. Several example elements will be described in reference to FIG. 8-FIG. 11 below.

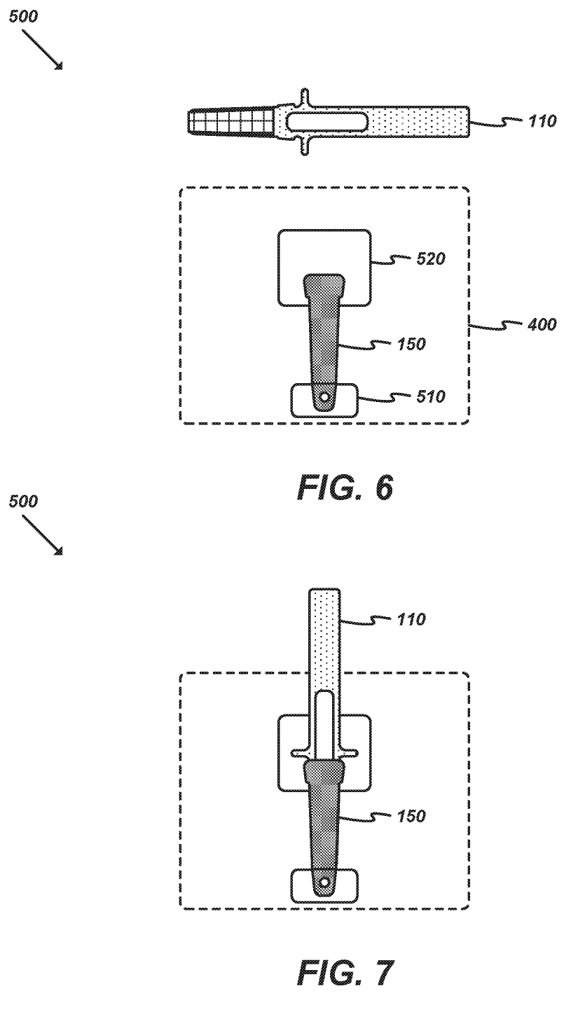

FIG. 6 illustrates a front sectional view of the measurement station 400 where the tester portion 110 has been separated from the base portion 150. In this example, a complete test cartridge 100 may be inserted into the base as shown in FIG. 5. Retention element 510 may then secure the base portion 150, such that the tester portion 110 may be removed in order to collect a sample.

FIG. 7 illustrates a front sectional view of the measurement station 400 where the tester portion 110 has been fully inserted into the base portion 150. In the fully inserted position, the coupling elements 140 may be engaged with the receptacles 160 such that the tester portion 110 and base portion 150 are firmly coupled together.

Continuing the above example, the tester portion 110 may be partially inserted as shown in FIG. 5. Manipulation element 520 may then move the tester portion 110 downward into the base portion 150. Such action may expose the test paper (and/or other sample collection feature) to the stored test solution (e.g., by breaching a reservoir such as reservoir 310). In some embodiments, the tester portion 110 may include a sharp edge or protrusion that is able to pierce an outer surface of the reservoir 310 in order to expose the sample collection element 210 to the solution stored in the reservoir 310.

FIG. 8 illustrates a side sectional view of the measurement station 400 including a first set of exemplary cartridge retention and manipulation elements. As shown, the base may include a support element 810, a first manipulation element 820, a second manipulation element 830, and one or more sensors 840.

The support element 810 may be a post as in this example, a portion of the housing 410, and/or include other appropriate elements (e.g., fasteners).

The first manipulation element 820 in this example includes a post that is able to engage the receptacle 170 of some embodiments. The second manipulation element 830 in this example includes two pairs of protruding arms (upper and lower arms on each side of the cartridge 100 as viewed from the front) that are able to engage the tabs 130 of some embodiments (and/or other appropriate features of the cartridge). The manipulation elements 820-830 of this example each pivot about an axis such that the cartridge 100 may be engaged and/or manipulated. The manipulation elements 820-830 may be associated with various springs, fasteners, motors, actuators, etc. that are able to manipulate and/or retain the position of the elements 820-830.

The sensors 840 may include optical sensors, position sensors, etc. that may be able to read data from the cartridge 100 (e.g., by scanning a graphic code) and/or otherwise sense attributes of the cartridge (e.g., by determining a relative position of the cartridge or a portion thereof).

FIG. 9 illustrates a side sectional view of the measurement station 400 including the first set of exemplary cartridge retention and manipulation elements after the cartridge 100 has been inserted. Such a state may be similar to the example of FIG. 5. The manipulation element 830 may be rotated in a clockwise direction in order to fully insert the tester portion 110 into the base 150 using the upper arms of the element 830.

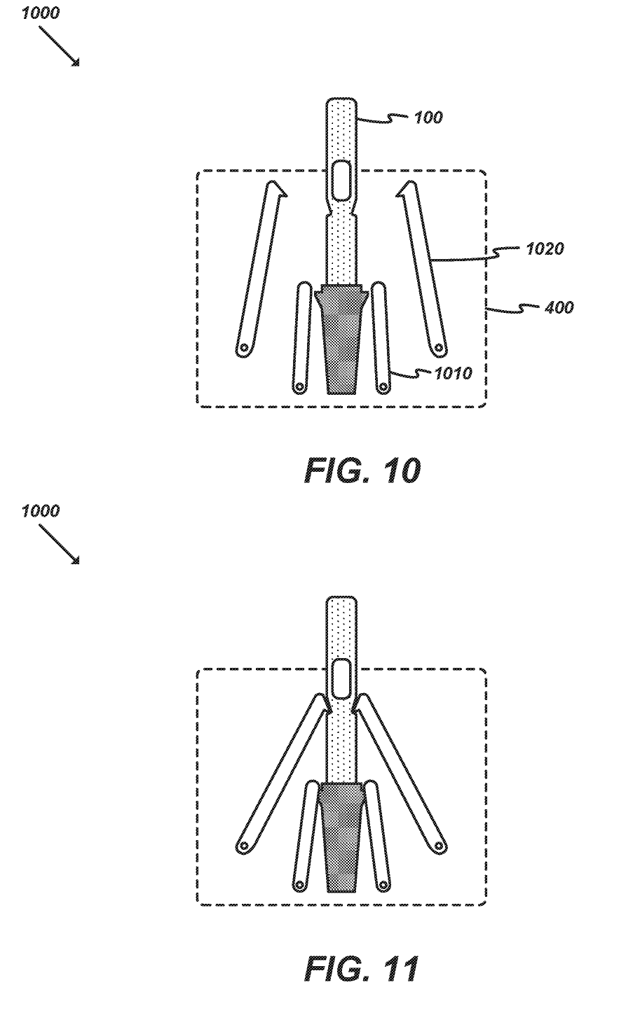

FIG. 10 illustrates a side sectional view of the measurement station 400 including a second set of exemplary cartridge retention and manipulation elements 1010-1020. This example includes a cartridge 100 having engagement features associated with the second set of exemplary cartridge retention and manipulation elements 1010-1020. As shown, the base 400 may include a set of base arms 1010 and a set of tester arms 1020. In this configuration, the cartridge 100 has been inserted into the base 400 but has not been engaged by the arms 1010-1020.

FIG. 11 illustrates a side sectional view of the measurement station 400 including the second set of exemplary cartridge retention and manipulation elements after the cartridge 100 has been inserted. As shown, the base arms 1010 may engage protrusions on the base portion 150 in order to retain the base portion. In addition, the tester arms 1020 may, after the tester 110 has been removed and a sample collected, engage the tester 110 such that the tester is fully inserted into the base 150 and the collected sample is exposed to the test solution in the reservoir 310.

One of ordinary skill in the art will recognize that the various elements described above may be implemented in various different ways without departing from the scope of the disclosure. For instance, the manipulation elements described above (and/or additional elements) may be used to eject a cartridge after a result has been obtained (or after some other appropriate criteria have been satisfied). As another example, some embodiments may include areas for cartridge (or reservoir) storage. Such storage may include storage for unused cartridges and storage for previously used cartridges that are intended for disposal. In addition, the housing 410 may include space for various electronic circuitry such as that described below.

### II. Hardware Architecture

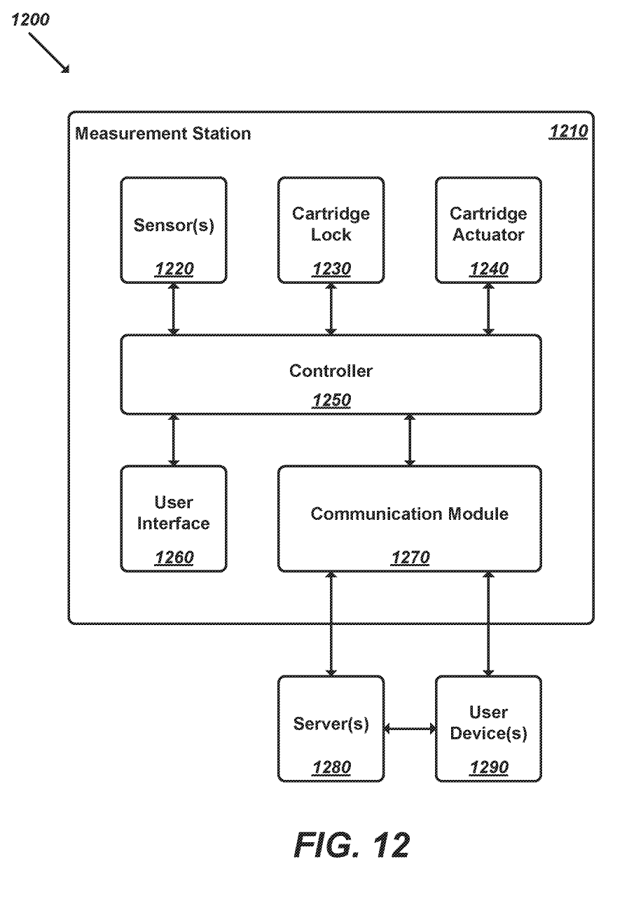

FIG. 12 illustrates a schematic block diagram of a system 1200 that provides sample management and evaluation according to an exemplary embodiment. As shown, the system may include a measurement station 1210 that has a set of sensors 1220, a cartridge lock 1230, a cartridge actuator 1240, a controller 1250, a UI module 1260, and a communication module 1270. In addition, the system 1200 may include one or more servers 1280 and/or user devices 1290.

The measurement station 1210 may be similar to those described above in reference to FIG. 4-FIG. 11.

The sensors 1220 may include, for instance, one or more cameras, motion sensors, pressure switches, environmental sensors, etc. The sensors may be used by the device 1210 to determine attributes of a cartridge (e.g., position, type, status, etc.). In addition, the sensors 1220 may be used to capture test results (e.g., by generating a color picture).

The cartridge lock 1230 may include various physical clamps, cams, actuators, etc., that are able to receive and retain a test cartridge 100 of some embodiments. The cartridge lock 1230 may be able to release the tester portion 110 of the cartridge 100 while retaining the reservoir portion 150 such that a sample may be collected before the tester 110 is returned to the measurement station 1210 (and coupled to the base 150) and activated. The cartridge lock 1230 may include elements such as manipulation features 820 and 1010.

In some embodiments, the measurement station 1210 may house a number of reservoir portions 150 (e.g., ten reservoirs may be housed in a reservoir pack) and a user may simply insert the tester portion 110 into an appropriate receptacle. The measurement station 1210 may then use the cartridge lock 1230, cartridge manipulator 1240, and/or other appropriate components to couple the tester portion 110 and the reservoir portion 150. In some embodiments, the cartridge lock 1230 and/or cartridge manipulator 1240 may include sub-elements for the reservoirs and/or testers. For instance, the cartridge lock 1230 may include a separate reservoir lock and tester lock. Likewise, the cartridge manipulator 1240 may include a separate reservoir manipulator and tester manipulator.

The cartridge actuator 1240 may include various motors, clamps, relays, etc. that may be able to push the tester portion into the reservoir portion such that the collected sample is exposed to (or submerged into) the testing solution stored in the reservoir. For instance, the cartridge actuator may include elements such as manipulation features 830 and 1020.

The controller 1250 may be able to direct, monitor, and/or otherwise interact with the various components of the measurement station 1210 and/or the cartridge. The controller 1250 may be able to execute instructions and/or otherwise process data. The controller may be associate with one or more local storages (not shown) that may be able to store instructions and/or other data.

The UI module 1260 may be able to generate various UI outputs and/or receive UI inputs. The module may be associated with various UI input elements (e.g., buttons, keypads, touchscreens, etc.) and/or output elements (e.g., display screens, indicator lights, haptic elements, audio outputs, etc.) The UI module may be able to provide instructions to users (e.g., “remove tester”, “insert tester”, “reload reservoirs”, etc.). In addition, the UI module may be used to provide test results. The UI module may also be able to modify or control the operations of the station 1210. For instance, after inserting a tester 110 a user may make a touchscreen selection or press a button indicating that the tester is ready for evaluation.

The communication module 1270 may be able to communicate across various networks and/or other appropriate pathways (e.g., local wired or wireless communication channels such as Bluetooth, Ethernet, etc.). The communication module be able to send and receive information such as test results, test parameters, biographic data, etc. The communication module may allow the measurement station to interact with various servers 1280 and/or user devices 1290. In some embodiments, the communication module 1270 may allow the UI module 1260 to interact with users via an external device (e.g., device 1290).

The servers 1280 may be electronic computing devices implemented as local devices, network accessible devices, distributed devices, etc. Such devices may be able to execute instructions and/or otherwise process data. The server may be associated with various local and/or remote storages. Some embodiments may include databases or other storage features that are able to be accessed by other devices (e.g., via an application programming interface).

The user devices 1290 may include devices such as smartphones, tablets, personal computers, wearable devices, etc. Such devices may be able to communicate across one or more networks and allow various users to interact with the station 1210. In addition, the server(s) 1280 and user device(s) 1290 may be able to interact with each other (and/or other resources).

Although system 1200 has been described with reference to various exemplary details, one of ordinary skill in the art will recognize that the system may be implemented in various different ways without departing from the scope of the disclosure. For instance, some embodiments may include additional devices or modules and/or omit various devices or modules. In addition, the devices or modules may be arranged in various different ways with various different communication pathways. As described above, various example embodiments may include various mechanical features that are able to manipulate, evaluate, and/or otherwise interact with a test cartridge.

### III. Methods of Operation

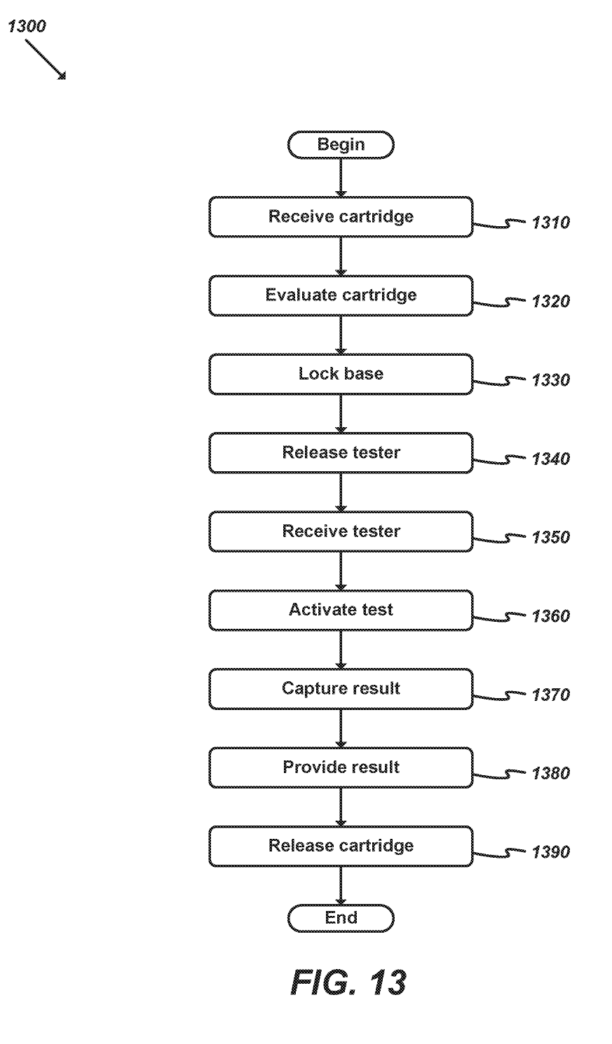

FIG. 13 illustrates a flow chart of an exemplary sample management and evaluation process 1300 implemented by the system and devices of FIG. 1-FIG. 12. Such a process may be executed by an element such as measurement station 110 described above. The process may begin, for instance, when the station 1210 is powered on and/or based on other appropriate conditions.

As shown, the process may receive (at 1310) a cartridge. Such a cartridge may include a tester portion (or a sample collection portion) and a reservoir portion (or evaluation compound portion). The tester portion 110 may include paper or other appropriate material that is able to be used to collect and store a sample. The reservoir portion 150 may include liquid that is able to interact with the paper in order to provide a result indication (e.g., by changing a color of at least a portion of the paper). In some embodiments, where the reservoirs are stored in the measurement station, the process may instead retrieve and/or otherwise position a reservoir 150 such that a tester 110 is able to be received.

The process may then evaluate (at 1320) the received cartridge 100. Such evaluation may include scanning a graphic code, bar code, or some other visually coded information with a camera or other appropriate sensor. Some embodiments may read a radio frequency ID or other transmitted identifying information.

In cases where the reservoir 150 is stored within the measurement station 1210, the cartridge attributes may already be known and simply retrieved from storage or otherwise obtained. In some embodiments, the process may interact with a server 1280 or other appropriate device such as a database in order to determine various characteristics, parameters, etc. associated with the received cartridge. Such information may include, for instance, cartridge or test type (e.g., pregnancy, blood sugar, etc.), possible results (e.g., associations of colors, patterns, etc. with a set of possible results such as positive, negative, within range, inconclusive, etc.), test parameters (e.g., wait time for valid result), and/or other appropriate information.

In addition to discrete result values, some embodiments may indicate a value within a range (e.g., one to ten, analog values represented by shaded or colored sections within a result area, etc.). Such a result may be determined by, for instance, scanning the result with a camera and analyzing the captured image data. The analysis may include conversion into various signals or values (e.g., an analog output, a digital code, etc.).

Next, the process may lock (at 1330) the base portion 150 of the cartridge 100 to the measurement station 1210. Such locking may be performed using various mechanical features, such as those described above.

The process may then release (at 1340) the tester portion 110 of the cartridge 100. In some embodiments, such “release” may only involve not securing the tester portion 110, where the tester portion and reservoir portion 150 may not be tightly coupled and able to be separated when the reservoir is locked in place. In the case where the reservoirs are housed by the measurement station, the tester 110 may be obtained from a tester supply or other appropriate resource. Some embodiments may utilize UI 1260 to provide an indication (e.g., by activating an LED, by modifying a display, by generating an audio signal, etc.) such that a user may be notified to retrieve the test in order to collect a sample.

Process 1300 may then receive (at 1350) the tester portion 110 of the cartridge 100 after a sample has been collected. The sample may be collected in various appropriate ways (e.g., swiping an area with the tester, applying sample liquid to an area of the tester, etc.).

The process may then activate (at 1360) the test. Such activation may involve, for instance, inserting the tester 110 into the reservoir 150 such that a seal or barrier is broken and interaction between the substance stored in the reservoir, the tester 110, and the collected sample.

After waiting any specified duration (and/or satisfying any other test criteria), the process may capture (at 1370) the test result. The result may be captured by sensors 1220. For instance, some embodiments may use a camera to generate a color photograph of a result indicator area of the tester.

Next, the process may provide (at 1380) the result. The result may be provided in various appropriate ways, depending on the specific situation. For instance, in some embodiments, the result may be displayed on a UI element 1260 of the measurement station 1210. As another example, the captured photograph and/or other information may be sent to a server 1280, user device 1290, mother station, etc. Provision of the result may involve various types of analysis (e.g., comparing a color (or other result indicator) to a result table to generate a discrete value for display to a user, performing visual recognition of a symbol or graphic code, etc.).

The process may then release (at 1390) the cartridge for disposal or other use and then may end. In addition, some embodiments may utilize UI 1260 to provide an indication (e.g., by activating an LED, by modifying a display, by generating an audio signal, etc.) such that a user may be notified to retrieve the used cartridge for disposal.

One of ordinary skill in the art will recognize that process 1300 may be implemented in various different ways without departing from the scope of the disclosure. For instance, some embodiments may perform the operations in different orders. As another example, some embodiments may include additional operations and/or omit listed operations. As still another example, some operations and/or sets of operations may be performed iteratively and/or based on some specified criteria.

### IV. Computer System

Many of the processes and modules described above may be implemented as software processes that are specified as one or more sets of instructions recorded on a non-transitory storage medium. When these instructions are executed by one or more computational element(s) (e.g., microprocessors, microcontrollers, digital signal processors (DSPs), application-specific integrated circuits (ASICs), field programmable gate arrays (FPGAs), etc.) the instructions cause the computational element(s) to perform actions specified in the instructions.

In some embodiments, various processes and modules described above may be implemented completely using electronic circuitry that may include various sets of devices or elements (e.g., sensors, logic gates, analog to digital converters, digital to analog converters, comparators, etc.). Such circuitry may be able to perform functions and/or features that may be associated with various software elements described throughout.

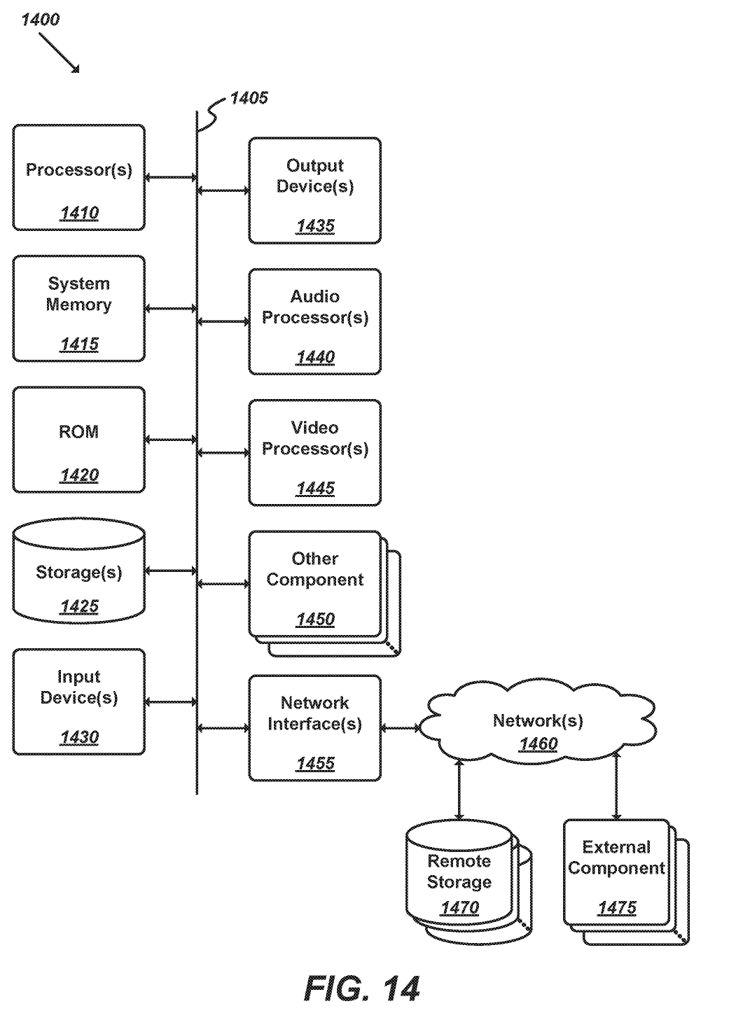

FIG. 14 illustrates a schematic block diagram of an exemplary computer system 1400 used to implement some embodiments. For example, the system and devices described above in reference to FIG. 1-FIG. 12 may be at least partially implemented using computer system 1400. As another example, the process described in reference to FIG. 13 may be at least partially implemented using sets of instructions that are executed using computer system 1400.

Computer system 1400 may be implemented using various appropriate devices. For instance, the computer system may be implemented using one or more personal computers (PCs), servers, mobile devices (e.g., a smartphone), tablet devices, and/or any other appropriate devices. The various devices may work alone (e.g., the computer system may be implemented as a single PC) or in conjunction (e.g., some components of the computer system may be provided by a mobile device while other components are provided by a tablet device).

As shown, computer system 1400 may include at least one communication bus 1405, one or more processors 1410, a system memory 1415, a read-only memory (ROM) 1420, permanent storage devices 1425, input devices 1430, output devices 1435, audio processors 1440, video processors 1445, various other components 1450, and one or more network interfaces 1455.

Bus 1405 represents all communication pathways among the elements of computer system 1400. Such pathways may include wired, wireless, optical, and/or other appropriate communication pathways. For example, input devices 1430 and/or output devices 1435 may be coupled to the system 1400 using a wireless connection protocol or system.

The processor 1410 may, in order to execute the processes of some embodiments, retrieve instructions to execute and/or data to process from components such as system memory 1415, ROM 1420, and permanent storage device 1425. Such instructions and data may be passed over bus 1405.

System memory 1415 may be a volatile read-and-write memory, such as a random access memory (RAM). The system memory may store some of the instructions and data that the processor uses at runtime. The sets of instructions and/or data used to implement some embodiments may be stored in the system memory 1415, the permanent storage device 1425, and/or the read-only memory 1420. ROM 1420 may store static data and instructions that may be used by processor 1410 and/or other elements of the computer system.

Permanent storage device 1425 may be a read-and-write memory device. The permanent storage device may be a non-volatile memory unit that stores instructions and data even when computer system 1400 is off or unpowered. Computer system 1400 may use a removable storage device and/or a remote storage device as the permanent storage device.

Input devices 1430 may enable a user to communicate information to the computer system and/or manipulate various operations of the system. The input devices may include keyboards, cursor control devices, audio input devices and/or video input devices. Output devices 1435 may include printers, displays, audio devices, etc. Some or all of the input and/or output devices may be wirelessly or optically connected to the computer system 1400.

Audio processor 1440 may process and/or generate audio data and/or instructions. The audio processor may be able to receive audio data from an input device 1430 such as a microphone. The audio processor 1440 may be able to provide audio data to output devices 1440 such as a set of speakers. The audio data may include digital information and/or analog signals. The audio processor 1440 may be able to analyze and/or otherwise evaluate audio data (e.g., by determining qualities such as signal to noise ratio, dynamic range, etc.). In addition, the audio processor may perform various audio processing functions (e.g., equalization, compression, etc.).

The video processor 1445 (or graphics processing unit) may process and/or generate video data and/or instructions. The video processor may be able to receive video data from an input device 1430 such as a camera. The video processor 1445 may be able to provide video data to an output device 1440 such as a display. The video data may include digital information and/or analog signals. The video processor 1445 may be able to analyze and/or otherwise evaluate video data (e.g., by determining qualities such as resolution, frame rate, etc.). In addition, the video processor may perform various video processing functions (e.g., contrast adjustment or normalization, color adjustment, etc.). Furthermore, the video processor may be able to render graphic elements and/or video.

Other components 1450 may perform various other functions including providing storage, interfacing with external systems or components, etc.

Finally, as shown in FIG. 14, computer system 1400 may include one or more network interfaces 1455 that are able to connect to one or more networks 1460. For example, computer system 1400 may be coupled to a web server on the Internet such that a web browser executing on computer system 1400 may interact with the web server as a user interacts with an interface that operates in the web browser. Computer system 1400 may be able to access one or more remote storages 1470 and one or more external components 1475 through the network interface 1455 and network 1460. The network interface(s) 1455 may include one or more application programming interfaces (APIs) that may allow the computer system 1400 to access remote systems and/or storages and also may allow remote systems and/or storages to access computer system 1400 (or elements thereof).

As used in this specification and any claims of this application, the terms “computer”, “server”, “processor”, and “memory” all refer to electronic devices. These terms exclude people or groups of people. As used in this specification and any claims of this application, the term “non-transitory storage medium” is entirely restricted to tangible, physical objects that store information in a form that is readable by electronic devices. These terms exclude any wireless or other ephemeral signals.

It should be recognized by one of ordinary skill in the art that any or all of the components of computer system 1400 may be used in conjunction with some embodiments. Moreover, one of ordinary skill in the art will appreciate that many other system configurations may also be used in conjunction with some embodiments or components of some embodiments.

In addition, while the examples shown may illustrate many individual modules as separate elements, one of ordinary skill in the art would recognize that these modules may be combined into a single functional block or element. One of ordinary skill in the art would also recognize that a single module may be divided into multiple modules.

The foregoing relates to illustrative details of exemplary embodiments and modifications may be made without departing from the scope of the disclosure as defined by the following claims.

I claim:

1. A medical test system comprising:
a test cartridge having a sample collection portion, a test substrate, a result indicator, and a reservoir portion that stores a test solution; and
a measurement station comprising:
a reservoir lock that couples to the reservoir portion of the test cartridge;
a tester manipulator that couples to the sample collection portion of the test cartridge and is able to insert the test substrate into the reservoir portion in order to apply the test solution to the test substrate; and
at least one sensor able to identify a result by evaluating the result indicator.

2. The medical test system of claim 1, wherein the test substrate comprises paper.

3. The medical test system of claim 1, wherein the result indicator is able to display at least one color.

4. The medical test system of claim 3, wherein the at least one sensor comprises a camera able to capture a color image of the result indicator.

5. The medical test system of claim 4, wherein evaluating the result indicator comprises determining a particular color provided by the result indicator and identifying a matching color from a result list.

6. The medical test system of claim 1 further comprising providing the result via at least one user interface element.

7. The medical test system of claim 1 further comprising transmitting the result to at least one external device.

8. A medical test cartridge comprising:
a tester portion including:
a test collection element;
a test substrate; and
a result indicator; and
a base portion coupled to the tester portion, the base portion comprising:
a reservoir that houses a test solution.

9. The medical test cartridge of claim 8, wherein the tester portion further comprises:
a set of tabs that are able to be engaged by a cartridge manipulation element; and
a set of coupling elements that secure the tester portion to the base portion.

10. The medical test cartridge of claim 9, wherein the base portion further comprises:
a receptacle that is able to be engaged by a cartridge retention element; and
a set of cavities that are able to accept the set of coupling elements when the tester portion is secured to the base portion.

11. The medical test cartridge of claim 8, wherein the test substrate comprises paper.

12. The medical test cartridge of claim 8, wherein the result indicator comprises a window that exposes a portion of the test substrate.

13. The medical test cartridge of claim 8, wherein the tester portion and the base portion are able to be coupled in a first configuration wherein the test substrate is not exposed to the test solution and a second configuration wherein the test substrate is exposed to the test solution.

14. The medical test cartridge of claim 8, wherein exposure of the test substrate to the test solution causes a visual indication to be provided via the result indicator.

15. An automated method of evaluating a medical sample, the automated method comprising:
receiving a base portion of a test cartridge;
securing the base portion using a retention element;
receiving a tester portion of the test cartridge; and
inserting the tester portion into the base portion.

16. The automated method of claim 15 further comprising:
identifying a cartridge type;
retrieving a set of test criteria associated with the cartridge type; and
retrieving a set of test results and associated indicators.

17. The automated method of claim 16 further comprising:
satisfying the set of test criteria;
capturing a result indicator;
identifying a test result based on the result indicator and the retrieved set of test results and associated indicators; and
providing the test result.

18. The automated method of claim 17, wherein providing the test result comprises sending the test result to a remote device.

19. The automated method of claim 17, wherein capturing the result indicator comprises capturing a color image of the result indicator and identifying the test result comprises matching a captured color to a particular associated indicator.

20. The automated method of claim 15 further comprising ejecting the test cartridge.
